(19) United States
(12) Patent Application Publication
(10) Pub. No.: US 2013/0052675 A1
(43) Pub. Date: Feb. 28, 2013

(54) TESTING SYSTEM FOR DETERMINING HYPOXIA INDUCED CELLULAR DAMAGE

(71) Applicants: Mathias Karlsson, Karlstad (SE); Sofia Hiort af Ornäs, Trosa (SE)

(72) Inventors: Mathias Karlsson, Karlstad (SE); Sofia Hiort af Ornäs, Trosa (SE)

(73) Assignee: CALMARK SWEDEN AKTIEBOLAG, Karlstad (SE)

(21) Appl. No.: 13/499,421

(22) PCT Filed: Sep. 30, 2010
(86) PCT No.: PCT/SE10/51048

§ 371 Date: Apr. 12, 2012

Related U.S. Application Data
(60) Provisional application No. 61/304,612, filed on Feb. 15, 2010.
(60) Provisional application No. 61/247,214, filed on Sep. 30, 2009.

(30) Foreign Application Priority Data
Sep. 30, 2009 (SE) 0950717-9

(51) Int. Cl. C12M 1/34; G01N 21/78

(52) U.S. Cl. 435/15; 435/288.7; 435/26; 436/129; 436/74

(57) ABSTRACT

The present invention relates to a testing system for assessing hypoxia induced cellular damage in a mammal including human, comprising a disposable device having a sample inlet and a collection chamber separated by a separation device wherein the collection chamber is connected to at least two, a first and a second, visible detection compartments, whereof at least one is arranged with chemical means for direct visual detection, said first detection compartment being arranged to determine whether level of hemoglobin (Hb) in a sample of body fluid taken from said mammal exceeds a predetermined threshold value, and said second detection compartment being arranged to evaluate level of total amount of lactate dehydrogenase (LDH) in said sample.

### TECHNICAL FIELD

The present invention relates to a testing system for assessing cellular damage, e.g. caused by hypoxia ischemia in a mammal including human comprising a disposable device having a sample collecting portion with a plasma separation device.

### BACKGROUND ART

Assessment of hypoxia (oxygen deficiency) in a mammal may be done by determining total lactate dehydrogenase (LDH) within body fluid obtained from a collected sample. Measuring total amount of LDH in combination with additional prognostic markers aspartate aminotransferase (AST), alanine aminotransferase (ALT) and lactate reveal status of mammal with respect to partial or complete oxygen deficiency, information which may underlie decisions of further medical actions. Examples of medical situations where detection of hypoxia is desirable are numerous, and include perinatal and neonatal monitoring of infants, triage in emergency rooms, surgery, transplantation or other medical procedures or surgical treatments. Obviously it is desired that detection of said biomarker/s is performed quickly so that adequate measures are taken as fast as possible to avoid permanent damages due to hypoxia.

A method of determining hypoxia is disclosed in U.S. application Ser. No. 12/101,470, where total LDH in plasma of a patient is measured, possibly in combination with either of K, Mg, Ca, AST, ALT and lactate, and where increased values of one or more of these markers is indicative of hypoxia in the patient. Also disclosed is the use of a plasma separation device in combination with an apparatus for quick quantitative and/or qualitative determination of mentioned markers. A way of determining prognostic marker levels according to U.S. Ser. No. 12/101,470 is by visual detection, arranged with dry chemical means.

Various other ways of measuring LDH levels are available, many of which are based upon visual detection caused by chemical reactions with reagents and dyes.

U.S. Pat. No. 4,056,485 finds utility in the determination of certain enzymes which causes reduction of colorless 2-(p-iodophenyl)-3-(p-nitrophenyl)-5-phenyltetrazolium chloride (INT) into bright red 1-(p-iodophenyl)-5-(p-nitrophenyl)-3-phenylformazan (INT formazan). The aqueous colored reference standard solution disclosed in U.S. Pat. No. 4,056,485 has an absorbance maximum at 500 nanometers and is suitable for use in the determination of for instance serum lactate dehydrogenase (LDH), creatine phosphokinase, glucose-6-phosphate dehydrogenase, adenosine phosphate, glucose, glucose-6-phosphate, 6-phosphogluconate and the like.

A general problem associated with today's methods for measuring biomarkers is that they often require access to a central laboratory having the possibility of measuring marker of interest, meaning the time to receive test results in some situations becomes undesirably long. In many places a central laboratory is not even available, and set-up of one would demand large investment costs.

An alternative to central labs is small measuring instruments, e.g. making use of a testing strip and measuring device such as a small foot-print instrument or hand-held spectrophotometer. Such equipments are expensive and commonly request a certain competence of the operator both to manage and interpret results of a reading. Looking at a global perspective many countries lack a properly functioning and advanced medical treatment system, and high technology solutions may not be applicable due to lack of economical resources, or simply because of lack of physicians or health care providers who are able to perform such tests.

Even in case of a developed medical care system there are situations where long lead time and/or complicated test apparatuses are not desirable, particularly if time is crucial and a mere indication of a medical status is enough for proceeding with adequate treatment of a patient.

In view of the foregoing there is a need for point-of-care testing methods applicable in various medical situations, where time is critical and quick assessment of patient status is of value for further medical treatment.

### OBJECTS OF THE INVENTION

It is a primary object of the present invention to provide an improved way of assessing hypoxia induced cellular damage during various medical situations, such improvement comprising the providing of a quick and user-friendly test, preferably a bedside-test, which is small, preferably independent of any instrument and which provides a way of nearly instant detection of elevated levels of selected biomarkers indicative of hypoxia in a mammal.

Additional objects of the invention will become evident from the following description and the claims.

### DISCLOSURE OF THE INVENTION

The object of the invention is achieved by a testing system for assessing hypoxia induced cellular damage in a mammal including human, comprising a disposable device with a sample inlet and a collection chamber arranged with a separation device wherein the collection chamber is connected to at least two (a first and a second) visible detection compartments, whereof at least one is arranged with chemical means for direct detection, said first detection compartment being arranged to determine whether the amount of hemoglobin (Hb) in a sample of body fluid taken from said mammal exceeds a predetermined level, and said second detection compartment being arranged to evaluate level of total amount of lactate dehydrogenase (LDH) in said sample by means of chemical means.

The object of the invention is also achieved by a method of assessing hypoxia induced cellular damage in a mammal, said method comprising the steps of providing a sample of body fluid from a mammal comprising particles such as blood cells, and subsequently separating said particles from said sample of body fluid by means of a separation device, contacting said separated body fluid with chemical means for direct detection, and determining if whether the amount of Hb in the body fluid is above or below a predetermined threshold value.

If the amount of Hb is below said threshold value the level of total amount of lactate dehydrogenase (LDH) in the body fluid is evaluated, and the risk for and/or presence of hypoxia induced cellular damage from the evaluation of the level of LDH in the body fluid.

In the present application “LDH” refers to the total amount of lactate dehydrogenase, not isoenzymes thereof.

The body fluid sample may be in the form of whole blood sample, serum, plasma, urine, cerebrospinal fluid (CSF), intraperitoneal fluid, or saliva, however the examples presented hereinafter are mainly related to testing of blood samples. It is to be understood that into the term “blood sample” may be interpreted other types of body fluids as previously mentioned.

By providing a microliter-volume blood sample and visually analyzing it with regards to chosen prognostic biomarkers using said invention, a testing system is provided which is quick, easy to use, easy to interpret, and which may be distributed as small stand-alone disposable units to medical practitioner who may use them in immediate connection to treating a patient, whether treatment is a surgical, triage or monitoring situation.

The term hypoxia means a partial or complete oxygen deficiency which may be caused by ischemia or inadequate oxygenation or severe anemia. Hypoxia may or may not lead to physical damage, and the body response to hypoxia differs depending on who the patient is. For instance in an infant subjected to hypoxia during or close to birth the body will redistribute the blood flow from “less important” organs in favor of the brain, heart and adrenals. An adult, on the other hand, may not tolerate the same level of hypoxia without damages. Hypoxia severe enough to damage cells will result in leakage of enzymes which enter circulation, and eventually cells will die further increasing enzyme concentration in the blood stream. Enzymes and prognostic markers that may be used to assess hypoxia induced cellular damage are LDH, aspartate aminotransferase (AST), alanine aminotransferase (ALT), lactate, creatine kinase (CK), K, Mg and Ca. In the present application the term hypoxia refers to oxygen deficiency severe enough to generate cellular damage.

According to one embodiment of the invention assessment of hypoxia induced cellular damage in a mammal is performed by first providing a blood sample from a mammal, including human, and applying the blood sample on the sample inlet of the testing system for separation of the red blood cells from the plasma through said plasma separation device. Next, a negative pressure inside said disposable device is generated for transferring the plasma through the plasma separation device and further into the at least first and second detection compartments where the plasma contacts reagents disposed therein. Each detection compartment is prepared with a reagent composition specific to a marker to be detected (e.g. Hb, LDH). Chemical reactions between marker in the plasma and the reagent composition (i.e. the chemical means) disposed in a detection compartment causes a visible color shift, meaning a colorimetric analysis is possible. For instance in case of Hb there may be a change in color if the level of Hb in the sample exceeds a certain predetermined level, otherwise no color shift will occur. Preferably in case of LDH, if the marker level is below a predetermined level, no color shift will occur in the corresponding detection compartment. If the marker level is above a predetermined level a color change will occur which is preferably, but not necessarily, proportional to the amount of the marker present in the plasma being tested. Each detection compartment is preferably visible, meaning an operator or health care provider will clearly see if a reaction is taking place therein and may thus visually determine presence of Hb and LDH respectively in the plasma. Presence of Hb above a predetermined level in a sample is indicative of hemolysis and since erythrocytes contains up to 150 times more LDH than blood serum hemolysis is a source of error. Thus in case of presence of Hb above said predetermined level the test needs to be remade. If no hemolysis has occurred the presence of hypoxia induced cellular damage is assessed from the visual colorimetric detection of LDH in the plasma.

According to one aspect of the invention a color change due to presence of a marker above a predetermined level may be interpreted by comparison with a standardized reference interval or color chart, calibrated to read quantitatively the amount of marker. For instance the amount LDH in a sample may be indicated in accordance with a standardized reference interval in the form of a scale presenting increasing color intensities, where less intense colors (e.g. light purple) correspond to lower concentrations of LDH and more intense colors (e.g. dark purple) correspond to higher concentrations of LDH. In accordance with the state of the art the color after reaction between the marker and the reagent composition is compared to the standard reference scale whereby levels of the marker in a sample may be assessed.

Obviously it is possible to provide a standardized reference interval presenting different colors, where for instance the color red indicates low concentration of the marker and purple indicates higher concentration.

According to another aspect of the invention the standardized reference interval is divided into a limited number of color sections, each section presenting a color density corresponding to a certain interval of the marker. In this way a step-wise based reference scale for assessing level of marker is attained, which may prove useful in situations where a more detailed information about the marker level is desired.

It is also within the scope of the present invention to measure the level of Hb by means of that the reagent composition, when contacted by a sample, gradually changes color, however that a color intensity corresponding to a preset threshold value of Hb is clearly indicated to safeguard that the test results are rightfully interpreted.

According to yet another aspect of the invention the detection compartment intended for assessment of hemolysis is free from any chemical means or reagents. Assessment of hemolysis is instead achieved merely by visually observing the filtered sample, preferably plasma, which is present within the detection compartment, and based on the color (or the hue) of said plasma determine whether hemolysis has occurred or not. Generally, if the plasma is transparent, no hemolysis has occurred, but if the plasma is pink or dark pink hemolysis can be suspected and the test should be remade. In order to facilitate assessment of hemolysis the testing system may be provided with a reference color chart intended for comparison with the color of the plasma, comprising a shade of pink indicative of hemolysis clearly demonstrated nearby the corresponding detection compartment. It is understood that such a reference color chart may be integrated with the disposable device but it may equally be provided as a free-standing part of the testing system delivered together with the disposable device.

In its most uncomplicated form the testing system of the invention may comprise a positive-negative reference only. According to such an embodiment a predetermined level is set for each marker, and the reagent composition within each detection compartment is arranged to shift/change color at said predetermined level so that a medical practitioner will simply know whether the amount of the chosen marker is below or equal to/above the preset level. Such a testing system may be advantageous when it is enough to indicate the risk of hypoxia induced cellular damage.

According to yet another aspect of the invention the detection compartments are arranged with chemical means in the form of dry chemical means or wet chemical means. According to one aspect of the invention each detection compartment is arranged with chemical means for a certain prognostic marker, such as LDH and Hb. Each detection compartment is prepared with a reagent composition arranged to react with one such marker. The reagent composition may be dry chemical means or wet chemical means depending on the design of a particular testing system.

According to one aspect of the invention the reagent composition for detection of LDH may comprise reagent in the form of tetrazolium compound, preferably selected from the group consisting of nitro blue tetrazolium (NBT), 1-(p-jodofenyl)-5-(p-nitrofenyl)-3-fenylformazan (INT) and 3-(4, 5-dimethylthiazol-2-yl)-5-(3-carboxymethoxyphenyl)-2-(4-sulfophenyl)-2H-tetrazolium (MTS), all of which are well known substances for colorimetric testing systems. Preferably reagent composition also comprises a mediator in the form of phenazine methosulphate (PMS) or 1-methoxy-5-methylphenazinium methylsulphate (mPMS) as well as and lactate and NAD⁺. According to one example the reagent composition for detection of LDH comprises tetrazolium compound (NBT) in a buffer comprising N-methyl-D-glucamine. Preferably the pH inside detection compartments is between 8-11, preferably between 8.5-10.5, even more preferably between 9-10.5 in order to optimize conditions for optimal enzyme reaction to take place. Reagent compositions for LDH is further illustrated in examples 1-4.

According to one aspect of the invention the reagent composition the reagent composition for detection of Hb may comprise reagent in the form of benzidine compound preferably selected from the group consisting of tetramethylbenzidine (TMB) and 3,3′-diaminobenzidine (DAB). The reagent composition for Hb may further comprise a peroxide substrate preferably selected from the group consisting of hydrogen peroxide, and tert-butylhydroperoxid (T-hydro). The pH inside the detection compartment 5A for Hb is preferably between 3-7, preferably between 4.5-5.5. Reagent compositions for Hb is further illustrated in examples 5-6.

According to one aspect of the invention wet chemical means are disposed within the disposable device inside storage arrangements for wet reagents, for example in reaction wells or in blister pack arrangements. According to one aspect, the blister pack arrangements are designed to rupture or be ruptured at initiation of use of the testing system, for instance by means of manual breakage before or after loading a sample onto the disposable device. Manual breakage may for instance be performed by a user pressing against the surface of the disposable device at a position which leads to compression of the blister pack and breakage thereof. Rupture of the blister pack results in that the chemical means is released and can be contacted by the sample to be tested. Thanks to this aspect the reaction between the reagent chemical components and the possible markers within the sample may be accelerated.

According to yet another aspect of the invention the disposable device comprises more than two detection compartments arranged on the card, preferably each one of said compartments arranged with chemical means in the form of a reagent composition. Preferably the more than two detection compartments each comprises chemical means for direct visual detection of one member of the group consisting of the following prognostic markers: Hb, LDH, aspartate aminotransferase (AST), alanine aminotransferase (ALT), lactate, creatine kinase (CK), K, Mg and Ca. Preferably each device comprises two detection compartments for detecting Hb and LDH respectively, and optionally one or more detection compartment for detection of one or more of AST, ALT, lactate, CK, K, Mg and Ca.

According to yet another aspect of the invention the testing system comprising means for generating a negative pressure inside said disposable device for urging the plasma from a blood sample to enter through said separation device and into the at least two detection compartments.

### BRIEF DESCRIPTION OF THE DRAWINGS

The testing system and the method of the invention will hereinafter be described in more detail with reference to the accompanying drawings. The following descriptions should be considered as preferred forms only, and are not decisive in a limiting sense.

FIG. 1A presents a schematic planar view of a testing system according to one example of the invention,

FIG. 1B presents a cross-sectional side view of a testing system according to FIG. 1A,

FIG. 1C presents a schematic planar view of a testing system comprising a subpressure generating device,

FIG. 2A-B presents the testing system according to another example of the invention,

FIG. 3 presents the testing system according to yet another example of the invention,

FIG. 4A presents a perspective view of a testing system according to an embodiment of the invention, having a separate capillary sample collector, and

FIG. 4B presents a perspective view of a testing system comprising an integrated capillary sample collector.

### DETAILED DESCRIPTION OF THE INVENTION

In the following detailed description reference will be made to the Figures illustrating various embodiments of the testing system 1 according to the invention. It is however to be understood that the invention also relates to a method for assessing hypoxia induced cellular damage in a mammal, and that many of the features which are disclosed in connection to the testing system 1 also are applicable to a corresponding method.

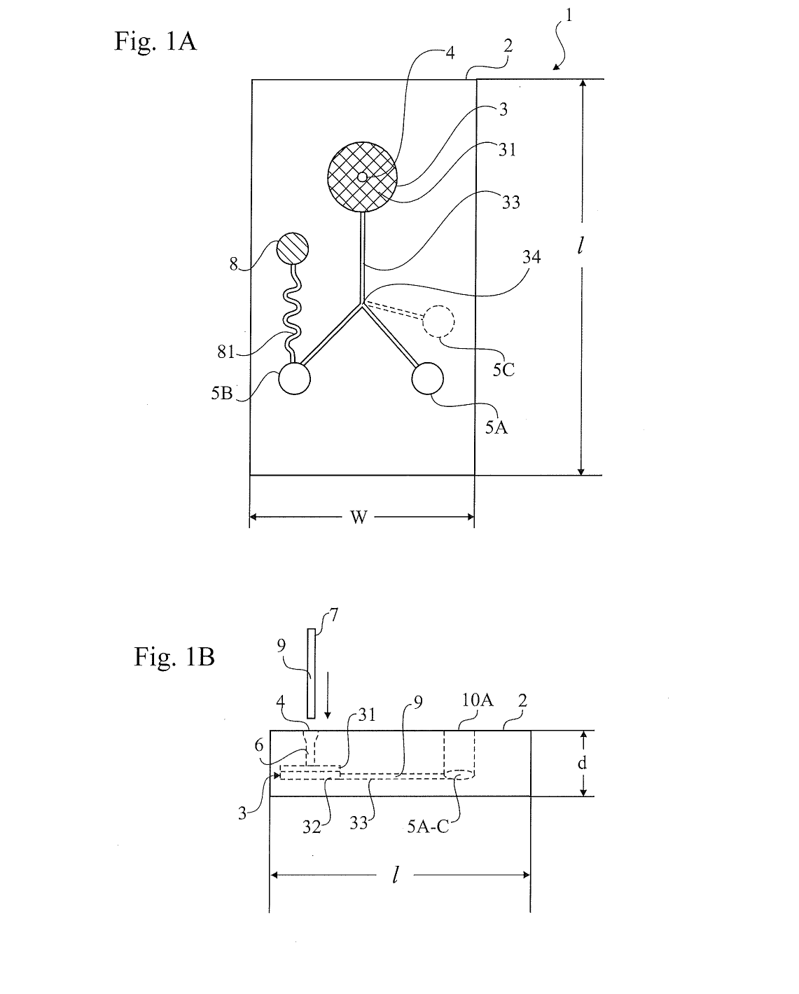

In FIGS. 1A-B there is shown a testing system 1 according to one embodiment of the invention including a disposable device 2, preferably arranged with a number of different detection compartments 5A-C as will later be explained in more detail. In FIG. 1A is schematically illustrated a planar view of the testing system 1 comprising a flat-shaped body here in the form of a cartridge device 2 having a sample collecting portion with a sample inlet 4 for receiving a sample of body fluid 9, e.g. whole blood, taken from a mammal. As schematically presented in FIG. 1B the disposable cartridge device 2 is provided with a receiving chamber 6 adapted to be fitted with a capillary sample collector 7 supplying a sample of body fluid 9 taken from a mammal. In connection with the receiving chamber 6, at the bottom thereof, there is an interface that in a manner known per se safeguards further transport of the body fluid sample to a separation device 3, the separation device comprising a filter 31 and a collection chamber 32. The filter 31 in FIG. 1A comprises the shape of a circle, and has an area of from 3 mm² to 500 mm², preferably less than 150 mm². It is understood that the suitable area of the filter 31 is depending on the desired sample volume, and that the filter area 31 therefore may be adjusted accordingly. The collection chamber 32 is connected, preferably via a microfluidic channel 33, to at least two, a first 5A and a second 5B, visible detection compartments whereof at least one, but possibly both, are arranged with chemical means for direct detection, preferably direct visual detection, at least of prognostic biomarker LDH. The detection 5A compartment arranged to determine the level or the amount of hemoglobin (Hb) in the sample 9′ may or may not be provided with chemical means. Hemolysis may be assessed by observing the hue of the plasma entering the corresponding compartment, in which case chemical means may not be necessary. It is however also possible to detect Hb with chemical means. In between the plasma collection chamber 32 and the detection compartments 5A-C the microfluidic channel 33 may be provided with a sample splitter 34 to direct plasma 9′ into each one of the different detection compartments 5A-C.

According to one example the first detection compartment 5A is arranged to determine whether the level of hemoglobin (Hb) in the sample of body fluid exceeds a predetermined level (a threshold value), and the second detection compartment 5B is arranged to evaluate the level of the total amount of lactate dehydrogenase (LDH) in said sample. As is indicated with dotted lines in FIG. 1A the disposable device 2 may include more than two detection compartments 5A-C connected to the collection chamber 32, wherein the compartments 5A-C comprise chemical means in the form of a reagent composition which will react with a prognostic marker, if present, so that a color-shift occurs, said color shift being within the visible spectrum so that it can be readily observed by the human eye. It is understood that the visible spectrum refers to the portion of the electromagnetic spectrum that can be detected by the human eye, typically ranging between 380 nm-750 nm.

According to the present invention the testing system 1 enables direct visual detection of a marker selected from the group consisting of Hb, LDH, aspartate aminotransferase (AST), alanine aminotransferase (ALT), lactate, CK, K, Mg and Ca. Indeed a device 2 testing LDH and Hb only may in some applications be sufficient.

However, it is also possible to detect results of a reading (i.e. a color shift) by means of spectrophotometric detection methods.

It is understood that samples of body fluids except for, or as a complement to blood sample may be readily analyzed using the testing system according to the invention, for instance urine, cerebrospinal fluid (CSF) or saliva. Said separation device 3 will clean the sample, separating undesired particles or sediments therefrom which may otherwise disturb the analysis.

As illustrated e.g. in FIGS. 1A-B, the disposable device 2 is in the form of a rectangular cartridge, however, the shape of device is not essential to the present invention, and persons of ordinary skill in the art can readily select a suitable shape or design for a given application. The device 2 may further be constructed from a material, such as transparent plastic, like cyclo-olefin (COC), polyethylene terephthalate (PET) or polymethyl methacrylate (PMMA) using a method such as injection molding or lamination. However, it is preferred that the device 2 is dimensioned so that it is portable and small enough to be able to be comfortably held in the hand of an operator. Said disposable device 2 is portable and has a length 1 between 3-15 cm, preferably 5-10 cm, a width W between 0.5-5 cm, preferably 2-4 cm and a thickness d between 0.1-3 cm, preferably 0.2-2.5 cm. Preferably said disposable device 2 has a weight between 5-50 g.

Use of the testing system 1 will now be described.

A sample of body fluid from a mammal, such as a whole blood sample, is first provided preferably, but not necessarily, by means of a capillary device 7 being filled with whole blood amounting to, e.g. about 50 μm. In a consecutive step the capillary device 7 is inserted into compartment 6 of the cartridge 2 to interface the blood sample 9 with the disposable device 2 placing the blood sample 9 onto the filter 31 of the plasma separation device 3. The skilled person understands that many ways of applying the fluid sample 9 on the cartridge 2 are possible, using a capillary sample collector 7 or other types of sample collectors. For instance a sample 9 may be applied directly onto the filter 31, e.g. by means of a sample collector in the form of a pipette releasing a sample volume thereon. Thus it is also understood that the design of the cartridge 2 may be such that the filter 31 is arranged at the upper surface of the cartridge 2, being exposed so that a sample volume 9 can be directly released thereon. A negative pressure is generated be means of a subpressure generating device 14 (see FIG. 1C) whereby the blood sample 9 is caused to be drawn through the filter 31 whereupon selected particles, particularly red blood cells, are filtered out. The serum (blood plasma) of sample passes through the filter 31 and is collected within collection chamber 32 and proceeds further through the micro fluidic channel 33 entering the different detection compartments 5A-C where reagents are deposited. Prognostic biomarkers present within the blood serum will react with deposited reagents causing a color-shift within the respective compartment, which can be detected by a user for assessing hypoxia induced cellular damage in the mammal (e.g. human) from whom the sample was collected.

Accordingly the method of the invention comprises the steps of:
providing a sample of body fluid from a mammal comprising particles such as blood cells,
separating said particles from said sample of body fluid by means of a separation device,
contacting said separated body fluid with chemical means for direct detection,—determining if whether the amount of Hb in the body fluid is above or below a predetermined threshold value, and if the amount of Hb is below said threshold value:
evaluating the level of total amount of lactate dehydrogenase (LDH) in the body fluid, and assessing the risk for and/or presence of hypoxia induced cellular damage from the evaluation of the level of LDH in the body fluid.

It is understood that the method according to the invention may be performed by means of a testing system 1 according to the invention (e.g. comprising a disposable device 2 with filter 31 and detection compartments 5A-C), but that other ways of performing the method are also conceivable. For instance it is foreseen that a medical practitioner may distribute filtered liquid sample 9′ in reaction wells and subsequently adding reagent composition which may for instance be delivered in single dose disposable containers.

In FIG. 1C there is illustrated one exemplary embodiment of the testing system 1 provided means 14 for generating a negative pressure inside said collection chamber 32 for urging the plasma from a sample of body fluid to pass through said separation device 3 and into the at least two detection compartments 5A-B.

According to one aspect of the invention said means 14 is manually manoeuvrable and arranged to generate a negative pressure inside the collection chamber 32 and the microfluidic channels 33, e.g. a compressable bellows pump 14 comprising a sealable vent hole 142. According the embodiment shown herein the subpressure generating device 14 is integrated with the cartridge 2 and is connected to the detection compartments 5A-C via microfluidic channels 141A, 141B. Generation of a negative pressure inside cartridge device 2 may be achieved in the following way. An operator pushes against the surface of the cartridge device 2 at a position corresponding to the location of the subpressure generating device 14, preferably indicated on the surface of the cartridge 2. Air will hereby exit from the microfluidic channels 33, 141A, 141B, 81 of the cartridge 2 via the sealable vent hole 142. Upon release of the subpressure generating device 14 the vent hole 142 is preferably sealed, e.g. by means of comprising a check valve, or in that the user manually seals the hole 142. This will lead to that release of the subpressure generating device 14 creates a subpressure inside the cartridge device 2 (for instance by a bellows pump retaking its original shape) and the fluids inside the microfluidic channels 33, 141A, 141B, 81 will hereby be urged to move through the testing system 1.

The disposable device is provided with optical viewing areas 10A-C through which corresponding detection compartments 5A-C can be observed, meaning a possible color-shift is readily observable by a user or health care provider. For Hb, it is preferred that a color-shift will occur only if level of Hb exceeds a predetermined level, said level being set as a threshold value, where values above the threshold indicate hemolysis. If a color-shift is observed in the compartment 5A for Hb, the test is invalid and a new test needs to be taken.

Regarding detection compartments other than for Hb a color-shift indicates presence of marker. Various solutions are possible regarding adaption of reagent composition and designing a standardized reference chart for interpretation of a possible color shift. According to one example the reagent composition is set to change or shift color only if marker is present above a predetermined concentration. Another option is that the reagent composition is set to gradually change color density for increasing concentrations, in which case color intensity is proportional to amount of marker present in the body fluid. Evidently it is possible that a detection compartment is colorless if marker level is below a preset limit, above which limit the color will appear more or less intense depending on concentration of marker.

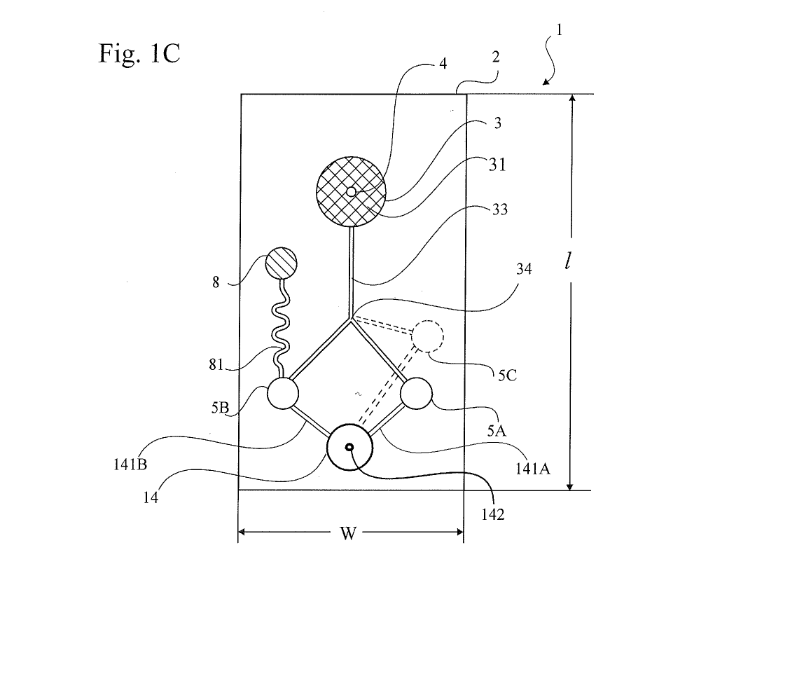

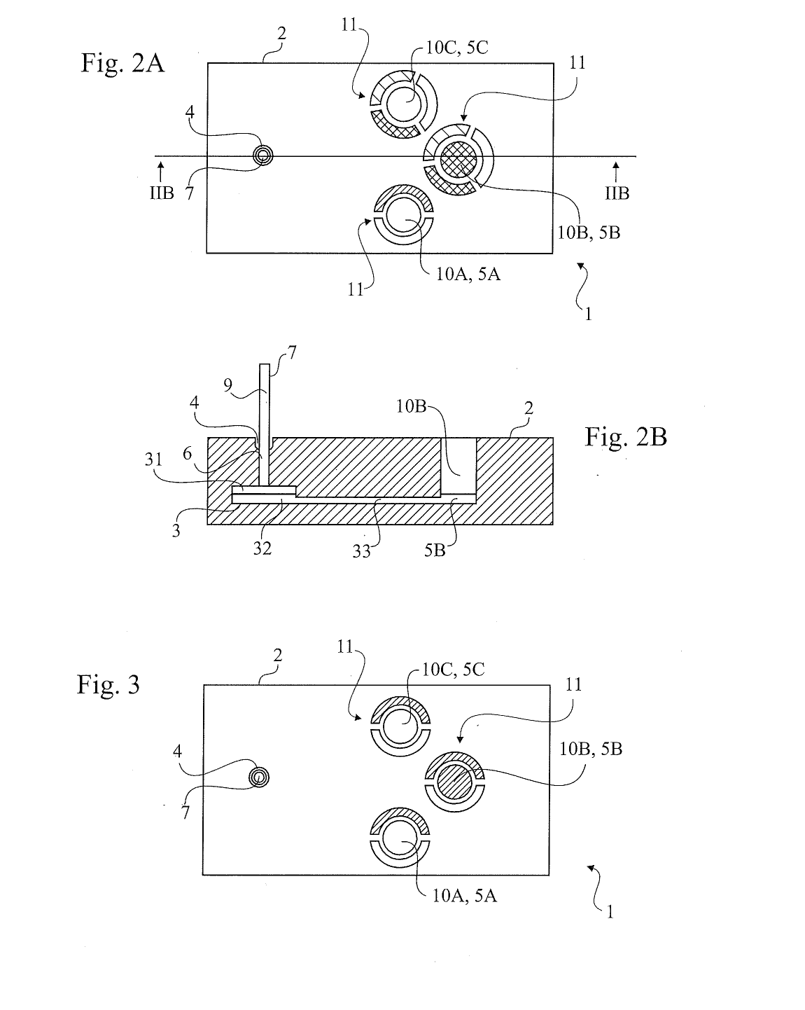

The intensity of the color-shift is compared to a standardized reference scale or interval whereby the level of the corresponding marker may be determined, and the risk of hypoxia assessed. The standardized reference scale may be designed in accordance with the adjustments of the reagent composition, as previously described herein, meaning it may be in the form of a number of discontinuous color sections, preferably at least two color sections, where the marker level is estimated by comparing a color-shift in any of the detection compartments with given color sections. The standardized reference scale is described in more detail in connection to FIGS. 2 and 3.

It is understood that the chemical means, deposited in the different detection compartments, may be in the form of dry chemical means or wet chemical means depending on the design of a particular testing system.

In case of wet chemical reagents, according to one exemplary embodiment of the invention, the reagent composition for each detection compartment may be placed within a protecting blister package located within the cartridge 2 in connection to each detection compartment 5A, 5B. In connection to initiation of testing by means of a system according to the invention the blister package is arranged to break, thus releasing the content in the form of said reagent composition. The introduced sample 9′ will thus mix with the wet reagent composition and reaction will commence, provided that the sample comprises the corresponding marker. Breakage of said blister package may be accomplished manually, for instance by means of a user pressing against a surface of the disposable device 2 so that the sides of the cartridge 2 is compressed enough to cause integrated blister to break.

Moreover in case of dry chemical means the chosen reagents are dried inside the detection compartments. When a fluid sample 9′ enters into the respective compartments the dried reagents will start to dissolve so that reaction can start. In order to facilitate rehydration of the reaction components it is preferred to add a supporting reagent to the dry chemical means.

As is previously stated chemical interaction between reagent composition within a detection compartment 5A-C and a marker causes a color-shift which alerts an operator of risk of hypoxia induced cellular damage. In order to safeguard robustness of testing system 1 it is desired that a reaction taking place inside a detection compartment 5A-C is limited to a predetermined time span so that all test units are comparable. For this reason the disposable device 2 may comprise a compartment 8 with reaction-stopper for interrupting the reaction between a biomarker and the reagent composition at a predetermined time after that a user of the test has generated the negative fluid pressure. This means that the time span from the point when a blood sample is first drawn through the filter 31 to when reaction-stopper interrupts the reaction between the reagent and the biomarker, is always the same.

As is evident to the skilled person it is possible to instead of a reaction stopper set a timer, and after a certain predetermined time assess whether any color changes have occurred.

An example of outline of a reaction-stopper 8 is seen in FIG. 1A where the disposable device 2 comprises a compartment 8 which contains a substance or compound suitable for interrupting enzymatic activity, for instance acid or basic solution like HCl, citric acid or NaOH. Further it is possible to use various surfactants or additives as reaction stopper, for instance sodium dodecyl sulphate (SDS) has proven to work well as a reaction stop.

According to one embodiment of the invention, when negative pressure is generated the reaction-stopper will start to flow through a micro fluidic channel 81 towards a detection compartment 5B in which it is intended to stop the reaction. The length of the microfluidic channel 81 will determine the time it takes for reaction-stopper to reach the compartment 5B. In FIG. 1A the microfluidic channel 81 is a serpentine-like channel for increasing the time before reaction is stopped, however many other ways of adjusting the length of channel 81 are equally possible.

Obviously it is equally possible to arrange the cartridge 2 so that the sample 9′ mixed with the reagent composition will move to a compartment arranged with a stationary reaction stopper 8 after a certain reaction time, e.g. via a microfluidic channel 81. In such an embodiment the stationary reaction 8 stopper may be in the form of dried or wet reaction stopper.

FIG. 1B shows, in a schematic way, a see-through side view of a disposable device 2 with sample inlet 4 in the form of a sample inlet connected to chamber 6 adapted to receive a capillary device 7 containing a whole blood sample 9 arranged to be placed onto plasma separation device 3. The sample inlet 4 is preferably surrounded by a funnel-like insertion pit for guiding a capillary sample collector 7 into chamber 6. Herein is further seen said optical viewing areas 10A which allow for observing ongoing reaction inside detection compartments 5A-B.

In FIG. 2A-B is presented an example of disposable device 2 according to the present invention. FIG. 2A is seen from a planar top-view, and FIG. 2B is a cross-view according to IIB in FIG. 2A. Herein device 2 is supplied with test blood 9 by means of a capillary device 7 being filled with whole blood amounting to, e.g. about 50 μL. Depending on the patient and/or on the particular design of device 2 (e.g. number of detecting compartments, size of the channels etc.) various amounts of blood sample are imaginable, and it is possible to use as little as 1 μL, or as much as 100 μL, a preferred amount being between 25-75 μL.

In order to facilitate insertion of sample the area around sample inlet 4 is preferably pitted for guiding capillary device 7 into chamber 6. In FIG. 2A the capillary device 7 has already been inserted into a compartment 6 of the cartridge 2 to interface the blood sample 9 with the cartridge 2 and placing the blood sample 9 onto the filter 31 of the plasma separation device 3. Instead of a capillary device 7 it is conceivable to provide the sample 9 by means of a pipette releasing a drop of sample onto a marked area on the cartridge 2. A negative pressure is manually generated and plasma is urged through the filter 31 and into plasma collection chamber 32 wherefrom it proceeds through microfluidic channel 33 and is distributed into different detection compartments 5A-C. As is seen in FIG. 2B the testing system comprises optical viewing areas 10B in that at least the portions 10A-C of the disposable device 2 above each detection compartment 5B is transparent, meaning each detection compartment 5B is visible and can be observed during ongoing reaction.

According to one embodiment each detection compartment 5A-C is prepared with a reagent composition arranged to react with one of the following prognostic markers: Hb, LDH, aspartate aminotransferase (AST), alanine aminotransferase (ALT), lactate, CK, K, Mg and Ca. Preferably each device 2 comprises at least two detection compartments 5A-B for detecting Hb and LDH respectively, and optionally one or more detection compartment for detection of one or more of AST, ALT, lactate, CK, K, Mg and Ca.

After a predetermined time-span the reaction is interrupted by the reaction stopper and any color-shift is visually detected by the user of the testing system 1. The total time from applying the blood sample 9 in 2A to determine test result in 2C is less than 5 minutes, but preferably within two minutes.

FIG. 2A presents a planar view of the testing system after that a possible reaction has taken place within detection compartments 5A-C. In order to determine the level of prognostic markers the color shift (if any) in each detection compartment 5A-C is compared to a standard reference interval which is preferably provided together with the testing system. According to one embodiment of the invention the area next to each detection compartment 5A-C is provided with a number of reference colors 11 whereby assessment of marker-level is easily performed. Here, detection compartment 5A is arranged to determine presence of Hb, and 5B-C are arranged to determine or estimate levels of any other prognostic marker (LDH, AST, ALT, lactate, CK, K, Mg or Ca).

For instance in FIG. 2A a situation is exemplified where no color-shift has occurred in the compartment for Hb 5A, indicating that the test is valid. A reaction has occurred in compartment 5B, which color-shift corresponds to one of given reference colors 11, whereas no notable reaction has occurred in compartments 5C. Preferably a user of a testing system 1 is instructed to react if color-shift has resulted in a certain color intensity. Such instructions may be marked in connection to the reference interval, for instance in the form of a symbol indicating the parts of reference interval representing risk of hypoxia.

In FIG. 2A the standard reference 11 for compartments 5B-C has three color sections, however a person skilled in the art will understand that a larger number of color sections is possible in order to increase resolution of a reading, as well as it is possible to have a continuous color interval instead of, as shown here, discontinuous color sections.

Yet another example of possible reference interval 11 is seen in FIG. 3 where a standard reference 11 has only two color sections, meaning a reading will provide a user with a positive or a negative answer only. Such a design of a reference standard is suitable in medical situations where it is possible to preset a concentration limit above which it is always required to take medical action, or in situations where a simple and fast reading is more important than a quantitatively precise measurement of the marker level.

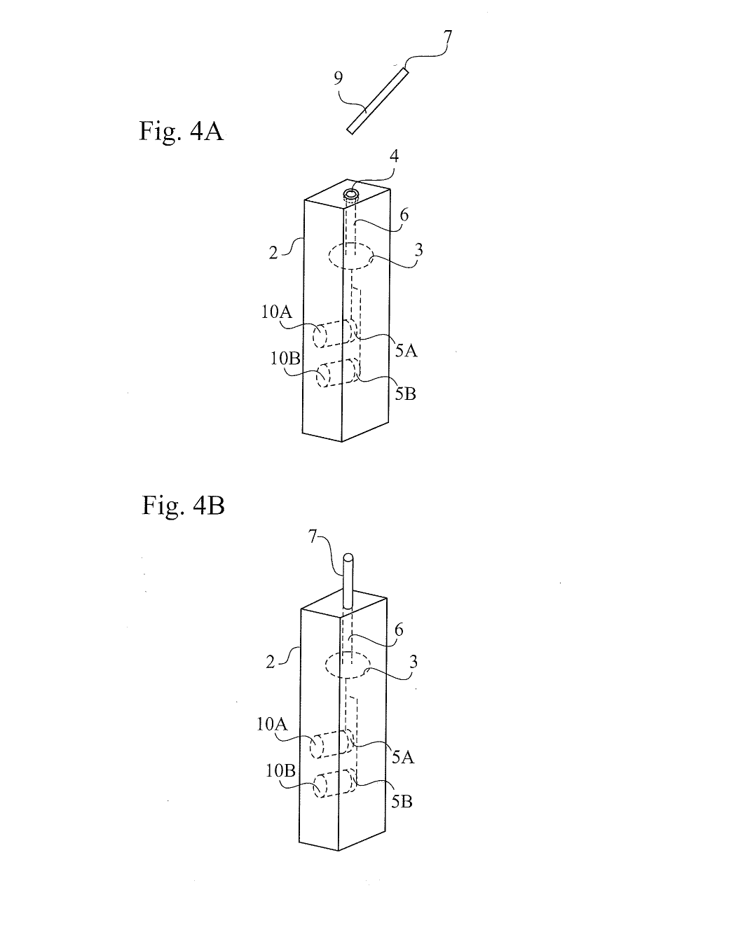

In 4A-B testing systems according to other examples of the invention are presented where instead of having a cartridge design the disposable device 2 is formed merely as a stick having a stretched-out body with two opposite short sides 21, 21′. According to the present example the sample inlet with sample inlet 4 is arranged at a short side 21 of the disposable device 2 (in connection to FIGS. 4A-B also referred to as “testing stick 2”).

Two designs of testing sticks 2 are illustrated herein. The first testing stick 2 schematically shown in FIG. 4A has a receiving portion with a chamber 6 similar to the one presented in FIGS. 1-2. A particular advantage with the chamber 6 of the testing stick 2 is that it may be arranged to accept the entire capillary device 7 so that no part of the capillary 7 extends outside of the stick 2 once it is inserted into chamber 6. Thus used testing sticks 2 may be disposed of as one entity which is favorable from a contamination perspective since no used and blood-containing capillary devices will be left unattended and accidentally break open.

The second testing stick 2 is illustrated schematically in FIG. 4B and comprises an integrated capillary member 7 protruding from one short end 21 and being in direct connection with the plasma separation device 3 inside the stick 2. A blood sample 9 may thus be collected directly into the device 2 with no need of handling the capillary 7 as a separate unit.

Beneficially, the method and the embodiments of the present invention allow for the determination of hypoxia in a wide variety of circumstances. For instance, embodiments of the present invention include, but are not limited to, the determination of hypoxia induced cell injury in a newborn baby by analyzing blood from the newborn baby, e.g. by analysing a sample provided from the umbilical cord.

The method and the embodiments of the present invention further may allow for determination of hypoxia in a gastrointestinal tract (e.g., colon anastomosis), specific organs (e.g., liver and aorta), cerebrospinal fluid from a lumbar drain, and organs to be transplanted. Additionally, embodiments of the present invention enable the assessment and/or monitoring of cellular leakage from one or more organ systems in a known and/or potentially critically ill patient (e.g., in mammal's potentially suffering from multi-organ dysfunction e.g., related to trauma, sepsis, haemorrhage or extensive surgery), prediction of brain injury after prenatal asphyxia (hypoxic ischemic encephalopathy, HIE), and monitoring of peripheral blood circulation of a mammal.

Different prognostic markers and combinations of prognostic markers have proven useful for assessing hypoxia in different medical situations as described in more detail in US2008/0213744, which is herewith incorporated in this application.

LDH is present in all body tissues and is a perfect marker of general cellular damage. However by combining with other markers the clinical picture could be even more clear. In the following is provided a few examples of combinations of markers which are of interest at particular medical situations.

LDH in combination of an organ specific marker like ALT (specific for liver).

LDH in combination with lactate and/or Mg that are more acute markers of on hypoxic event taking care generally in the whole organism or in a specific tissue.

LDH in combination with AST and ALT, all with different half life in plasma making the combination a potential temporal marker of an hypoxic event that have occurred earlier. In a medico legal aspect when a retrospective investigation is taking place when a newborn infant is damage by asphyxia.

Hereinafter a few medical conditions where hypoxia is a serious concern are presented and testing system according to the present invention thus would be beneficial.

During or close to birth, assessment of hypoxia allows for prediction of perinatal asphyxia and/or brain injury after prenatal asphyxia (e.g. HIE). In a situation of predicted brain injury the newborn child is provided hypothermia treatment whereby development of hypoxic ischemic encephalopathy (HIE) may be avoided. Hereby is provided a quick and easy way of identifying infants who are in danger of developing HIE, something which could save countless children from brain damage, especially in countries where medical care systems are presently not advanced enough to identify these infants.

During a triage situation the goal is to sort waiting patients so that the most urgent cases are treated first. Assessment of hypoxia by measuring one or more of presented markers is one way of being able to sort patients in a waiting room.

In one embodiment, a first reference blood sample is collected from a location of interest prior to a medical procedure and analyzed for prognostic markers using the testing system according to the present invention. Prior to completion of the medical procedure, a second blood sample can be obtained from the point of interest and analyzed in the same manner as the initial sample. The determination of prognostic markers in first and second samples can be compared in order to assess the presence of hypoxia induced cellular damage. In various embodiments, multiple prognostic markers are analyzed.

Such embodiments comprise determining amount of at least Hb and LDH in the plasma of both reference and final blood samples, and optionally one or more additional prognostic marker selected from the group consisting essentially of K, Mg, Ca, AST, ALT, CK and lactate. Accordingly, the respective amounts of each prognostic marker in the first and second samples can be compared to identify a proper location for an anastomosis. In one embodiment, the medical procedure comprises anastomosis of the gastrointestinal tract.

In another aspect, embodiments of the invention can reduce the morbidity and mortality rates in patients after transplantation therapy. One of the key factors impacting morbidity and mortality rates in patients after transplantation is related to preservation injury of grafts, such as the hepatic grafts in a liver transplant. For example, LDH, AST and ALT leakage into the perfusate is an indication of loss of the membrane integrity of the liver cells.

In one such embodiment, the method for determining the presence of hypoxia induced cellular damage in an organ to be transplanted into a mammal in need thereof can comprise providing a blood sample and analyzing the sample, as described above, for prognostic markers prior to the transplantation surgery. In one embodiment, the sample is analyzed to determining presence of Hb and the total amount of LDH and at least one additional prognostic marker in the sample selected from the group consisting essentially of K, Mg, Ca, AST, ALT, CK and lactate. In one preferred embodiment, the organ for transplant comprises a liver.

In yet another aspect, embodiments of the present invention can be used to assess the status of a mammal's limbs before and after medical or surgical treatment. For instance, trauma, fractures and vessel occlusions can affect the circulation to peripheral limbs and muscles (e.g compartment syndrome). As also presented in US2008/0213744 there exists a significant correlation between oxygen in ischemic muscle and levels of lactate and LDH, and lactate is elevated in femoral blood in patients with peripheral arterial occlusive disease compared to control values. Devices according to embodiments of the present invention make it possible to use enzyme and lactate levels to diagnose ischemia of a specific limb and also to assess the effects of most treatments.

Additionally, embodiments of the present invention comprise a method for determining hypoxia-ischemic by analyzing a sample from a limb of interest and determining the total amount of LDH in the plasma. Also the border for viable tissue during amputation of a limb could be assessed during surgery using the device Additional prognostic markers can be quantified at the same time as the determination of LDH. This allows an assessment of blood circulation to a mammal's limbs before and after a medical or surgical treatment.

Embodiments of the present invention include a device and a method for determining hypoxia induced cellular damage bedside, wherein the results are available within a matter of a few minutes at most. Such embodiments include obtaining a sample for analysis and determination of Hb and LDH. In preferred embodiments, the method include determining the amount of at least one additional prognostic marker in the plasma selected from the group consisting essentially of AST, ALT and lactate.

The reagent compositions for LDH and Hb respectively are further described by the following non-limiting examples 1-6, wherein 1-4 relate to detection of LDH and 5-6 relate to detection of Hb.

### Example 1

Tetrazolium salts, nitro blue tetrazolium (NBT), 2-p-iodophenyl-3-p-nitrophenyl-5-phenyl tetrazolium chloride (INT) and 3-(4,5-dimethylthiazol-2-yl)-5-(3-carboxymethoxyphenyl)-2-(4-sulfophenyl)-2H-tetrazolium (MTS) were dissolved separately in dimethyl sulphoxide producing 10 mM stock solutions. The mediators phenazine methosulphate (PMS) and 1-methoxy-5-methylphenazinium methylsulfate (mPMS) were dissolved separately in water producing 1 mM stock solutions. Stock solution of NAD was prepared in buffer. Sodium lactate was dissolved in water, and pH was adjusted to about 9 with 1 M tris.

Control sera (2.2 and 4.7 μkatal/l respectively) and blood sample from co-worker were used.

Blood samples were collected in Li-heparin tubes with separator (Vacuette, Greiner) and potassium-EDTA tubes (Vacuette, Greiner). The tubes were centrifuged for 15 minutes at 1500×g and plasma was transferred into Eppendorf tubes.

Enzyme assay was performed using conventional spectrophotometer using a Shimadzu UV-VIS 1610 spectrophotometer using plastic 1 ml cuvettes, in addition to visual inspection. The reaction mixtures were prepared according to table 1, and reactions were initiated by the addition of 50 μl NAD.

TABLE 1
Reaction mixture

| | |
|---|---|
| Buffer Tris/HCl | 800 μl |
| Tetrazolium stock (10 mM) | 50 μl |
| Mediator stock (1 mM) | 50 μl |
| Lactate stock (0.8M) | 50 μl |
| Sample (plasma or control serum) | 200 μl |
| NAD⁺ | 50 μl |

Results

The NBT appeared dark blue, INT purple and MTS redish-brown after the reactions, yielding a shift in color after a certain time of the reaction.

### Example 2

Assays were performed using an ELISA plate reader from Emax Molecular Devices, using 96-well plates in addition to visual inspection. The bottom of the 96-well pates is used as an optical surface for measurement and each well can contain up to 400 μl liquid. Absorbance will vary depending on solution depth in wells. Plates used in this experiment were from NUNC (high binding capacity).

Tetrazolium salts, nitro blue tetrazolium (NBT), 2-p-iodophenyl-3-p-nitrophenyl-5-phenyl tetrazolium chloride (INT) and 3-(4,5-dimethylthiazol-2-yl)-5-(3-carboxymethoxyphenyl)-2-(4-sulfophenyl)-2H-tetrazolium (MTS) were dissolved separately in dimethyl sulphoxide producing 10 mM stock solutions. The mediators phenazine methosulphate (PMS) and 1-methoxy-5-methylphenazinium methylsulfate (mPMS) were dissolved separately in water producing 1 mM stock solutions. Stock solutions of NAD and NADH were prepared in buffer. Sodium lactate was dissolved in water, and pH was adjusted to about 9 with 1 M tris.

Control sera (2.2 and 4.7 μkatal/l respectively) and blood sample from co-worker were used.

Blood samples were collected in Li-heparin tubes with separator (Vacuette, Greiner) and potassium-EDTA tubes (Vacuette, Greiner). The tubes were centrifuged for 15 minutes at 1500×g and plasma was transferred into Eppendorf tubes.

Measurement of enzyme activity was done in a total volume of 100 and 50 μl respectively.

TABLE 2
Reaction mixture for 100 μl reaction volume:

| | |
|---|---|
| Buffer Tris/HCl | 5 μl |
| Tetrazolium stock (10 mM) | 5 μl |
| Mediator (1 mM) | 5 μl |
| Lactate stock (0.8M) | 5 μl |
| Sample | 75 μl |
| NAD⁺ | 5 μl |

The sample was also tested as diluted when using blood sample, corresponding to less than 20 μl plasma.

TABLE 2
Reaction mixture for 50 μl reaction volume:

| | |
|---|---|
| Sample | 37.5 μl |
| Reaction mixture | 12.5 μl |

The sample was also tested as diluted when using blood sample, corresponding to less than 10 μl plasma.

Reaction mixture: Equal volumes of tetrazolium salt stock solution, mediator stock solution, lactate and NAD⁺ stocks were mixed prior to adding to sample.

Results

Shifts in color were successfully observed for all tetrazolium salt dyes.

NBT is well suited for visual detection of LDH activity. Both PMS and mPMS can serve as mediators, however mPMS is preferable since it is less sensitive to photochemical decomposition. Surprisingly enough the examples show that small volumes, even below 10 μL, are sufficient for giving a color shift acceptable for visual detection.

### Example 3

The following example 3 relates to wet reagent composition for assessing presence of LDH in a plasma sample.

Tetrazolium salt, nitro blue tetrazolium (NBT), was dissolved in dimethyl sulphoxide producing 10 mM stock solution. The mediator 1-methoxy-5-methylphenazinium methylsulfate (mPMS) was dissolved separately in water producing 1 mM stock solutions. Stock solution of NAD⁺ was prepared in buffer. Sodium lactate was dissolved in water. N-methyl-D-glucamine was dissolved in water (1M) and pH adjusted to 10 with HCl.

Control sera (2.2 and 4.7 μkatal/l respectively) and blood sample from co-worker were used.

Blood samples were collected in Li-heparin tubes with separator (Vacuette, Greiner) and potassium-EDTA tubes (Vacuette, Greiner). The tubes were centrifuged for 15 minutes at 1500×g and plasma was transferred into Eppendorf tubes.

Reaction mixture: equal volumes of tetrazolium salt stock solution, mediator stock solution, lactate and NAD⁺ stocks were mixed prior to adding to sample.

Measurement of enzyme activity was done in a total volume of 100 μl with 80 μl reaction mixture and 20 μl sample.

Results

Shifts in color were successfully observed. Color change was detected visually and spectrophotometrically.

### Example 4

The following example 4 relates to dry reagent composition for assessing presence of LDH in a plasma sample.

Stock solutions according to table 4 were prepared.

TABLE 4
Reaction mixture

NBT (10 mM)
mPMS (2.5 mM)
NAD (50 mM)
L-lactate (5M)
NMG (2M)

Using the stock solutions, NMG (0.61 ml), NAD (0.467 ml), L-lactate (0.440 ml), NBT (1.21 ml) and mPMS (0.997 ml) were mixed and dried onto a plastic sheet.

After drying, 5 μL of LDH-spiked plasma was applied on the dry spot and reaction was allowed to proceed for 2 minutes. The reaction was stopped with 2 M HCl.

Results

Color delineation between high and low LDH levels was clearly observed.

### Example 5

The following example 5 relates to wet reagent composition for assessment of Hb in plasma.

Reagent solutions were prepared as follows. Chromogenic compounds, N,N,N′,N′-Tetramethylbenzidine (TMB) and 3,3′-diaminobenzidine (DAB) were dissolved separately in dimethyl sulphoxide or directly in buffer solution (phosphate-citrate buffer). The substrates hydrogen peroxide, and tert-butylhydroperoxid (T-hydro) were dissolved separately in the respective chromogenic compound solutions. The pH was adjusted in the range 4-7.

A volume of 90 μl of the reagent solution and 10 μl of plasma (sample) were mixed for reaction.

Results

Color was developed successfully for each of the respective reagent solutions. TMB shifted color from transparent yellow (no Hb present) to green (Hb present). DAB shifted color from transparent (no Hb present) to brown (Hb present). Color change was detected visually and spectrophotometrically.

### Example 6

The following example 6 relates to dry reagent composition for assessment of Hb in plasma.

A reagent mixture consisting of TMB, hydrogen peroxide and buffer (pH 5.5) was dried onto a plastic sheet and rehydrated by a 10 μl plasma sample containing spiked levels of Hb. After rehydration the concentration of TMB was 0.2 mg/ml, hydrogen peroxide 0.04% and buffer 50 mM.

Results

The color development from the run showed a good delineation for samples with different concentrations of Hb, with an increased color density at higher concentrations of Hb.

The skilled person realizes that a large variety of modifications may be performed without the use of inventive skill, departing from the description above, e.g. the use of glass or some other suitable material in place of plastic etc. Furthermore it is within the scope of the present invention to analyze the test results (color shifts) by means of spectrophotometric methods within the visible spectrum.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1-54. (canceled)

55. Testing system for assessing hypoxia induced cellular damage in a mammal including human, comprising a disposable device (2) with a sample inlet (4) and a collection ch (32) arranged with a separation device (3) characterized in that the collection chamber (32) is connected to at least two, a first (5A) and a second (5B), visible detection compartments, whereof at least one is arranged with chemical means for direct detection, said first detection compartment (5A) being arranged to determine whether the amount of hemoglobin (Hb) in a sample of body fluid (9) from said mammal exceeds a predetermined level, and said second detection compartment (5B) being arranged to evaluate level of total amount of lactate dehydrogenase (LDH) in said sample by means of said chemical means.

56. Testing system according to claim 55, wherein the at least two detection compartments (5A, 5B) are arranged with means for direct detection, wherein said means for direct detection is any of chemical means for direct detection of said amounts of Hb and LDH respectively, chemical means for direct visual detection by means of colorimetry or chemical means for direct detection by spectrophotometric means.

57. Testing system according to claim 55, wherein:
said sample is selected from the group consisting of whole blood, plasma, serum, urine, cerebrospinal fluid (CSF), intraperitoneal fluid, and saliva; and
in case said sample is whole blood, said separation device (3) comprises a filter (31) for separating plasma (9′) from blood cells within said whole blood sample (9).

58. Testing system according to claim 55, wherein the volume of the sample of body fluid (9) is from 1 μL-100 μL.

59. Testing system according to claim 58, wherein the volume of the sample of body fluid (9) is from 10 μL-75 μL.

60. Testing system according to claim 55, wherein the total amount of a prognostic marker in the sample of body fluid (9) is estimated by comparison to a standard reference scale of increasing color intensity, whereas absence of color or less intense color corresponds to low concentration of marker and more intense color corresponds to high concentration of marker.

61. Testing system according to claim 55, wherein the disposable device (2) comprises more than two visible detection compartments (5A-C) arranged on the card, preferably each one of said compartments arranged with chemical means in the form of a reagent composition, wherein each of said visible detection compartments (5A-C) is arranged with reagent composition for direct visual detection of one member of the group consisting of prognostic markers Hb, LDH, aspartate aminotransferase (AST), alanine aminotransferase (ALT), lactate, creatine kinase (CK), K, Mg and Ca.

62. Testing system according to claim 55, wherein said sample inlet (4) comprises an integrated capillary sample collector (7) for collecting a sample of body fluid from a mammal.

63. Testing system according to claim 55, wherein said chemical means is a reagent composition in the form of dry chemical means or wet chemical means, wherein said reagent composition is arranged to determine LDH comprising tetrazolium compound, said compound being selected from the group consisting of nitro blue tetrazolium (NBT), 1-(p-jodofenyl)-5-(p-nitrofenyl)-3-fenylformazan (INT) and 3-(4, 5-dimethylthiazol-2-yl)-5-(3-carboxymethoxyphenyl)-2-(4-sulfophenyl)-2H-tetrazolium (MTS) and wherein said reagent composition further comprises a mediator in the form of at least one of phenazine methosulphate (PMS) and 1-methoxy-5-methylphenazinium methylsulphate (mPMS), wherein said reagent composition further comprises lactate and NAD⁺.

64. Testing system according to claim 63, wherein said reagent composition is arranged to determine LDH in a buffer comprising N-methyl-D-glucamine, and further wherein the pH inside the detection compartments (5A-C) is between 9-11.

65. Testing system according to claim 64, wherein the pH inside the detection compartments (5A-C) is between 9.5-10.5.

66. Testing system according to claim 65, wherein the pH inside the detection compartments (5A-C) is between 9.8-10.2.

67. Testing system according to claim 55, wherein said chemical means is a reagent composition in the form of dry chemical means or wet chemical means, wherein said reagent composition is arranged to determine Hb, comprising a benzidine compound selected from the group consisting of tetramethylbenzidine (TMB) and 3,3′-diaminobenzidine (DAB), wherein said reagent composition comprises a peroxide substrate selected from the group consisting of hydrogen peroxide, and tert-butylhydroperoxid (T-hydro), wherein the pH inside the detection compartment (5A) for determining Hb is between 3-7.

68. Testing system according to claim 67, wherein the pH inside the detection compartment (5A) for determining Hb is between 4.5-5.5.

69. Testing system according to claim 55, wherein said disposable device (2) comprises a compartment (8) with reaction-stopper for interrupting reaction between a prognostic marker and said reagent composition after a predetermined time span.

70. Testing system according to claim 55, the system further comprising means (14) for generating a negative pressure inside said collection chamber (32) for urging the plasma from a sample of body fluid to pass through said separation device (3) and into the at least two detection compartments (5A-B).

71. Testing system according to claim 55, wherein said disposable device (2) is portable and has a length (l) between 3-15 cm, a width (W) between 0.5-5 cm, and a thickness (d) between 0.1-3 cm, wherein said disposable device (2) further has a weight between 5-50 g.

72. Testing system according to claim 71, wherein said length (l) is between 5-10 cm.

73. Testing system according to claim 71, wherein said width (W) is between 2-4 cm.

74. Testing system according to claim 71, wherein said thickness (d) is between 0.3-0.7 cm.

75. An in-vitro method of assessing hypoxia induced cellular damage in a mammal, said method comprising the steps of:
providing a sample of body fluid (9) from a mammal comprising particles such as blood cells;
separating said particles from said sample of body fluid (9) by means of a separation device (3);
contacting said separated body fluid (9′) with chemical means for direct detection;
determining if whether the amount of Hb in the body fluid (9′) is above or below a predetermined threshold value, and if amount of Hb is below said threshold value;
evaluating the level of total amount of lactate dehydrogenase (LDH) in the body fluid (9′); and
assessing the risk for and/or presence of hypoxia induced cellular damage from the evaluation of the level of LDH in the body fluid (9′).

76. The method according to claim 75, wherein, in steps (d)-(e), determining and evaluating levels of markers Hb and LDH respectively, are performed by direct visual detection via at least one of colorimetry and spectrophotometric detection.

77. The method according to claim 76, wherein:
the sample is a body fluid (9) selected from the group consisting of: whole blood, plasma, serum, urine, cerebrospinal fluid (CSF), intraperitoneal fluid, and saliva; and
said separation device (3) comprises a filter (31) for separating plasma (9′) from blood cells within said whole blood sample (9).

78. The method according to claim 77, wherein the volume of the sample of body fluid (9) is from 1-100 μL.

79. The method according to claim 78, wherein the volume of the sample of body fluid (9) is from 10 μL-75 μL.

80. The method according to claim 75, wherein:
said chemical means is a reagent composition in the form of dry chemical means or wet chemical means, and wherein said reagent composition is arranged to determine LDH, and comprises a tetrazolium compound, said compound being selected from the group consisting of: nitro blue tetrazolium (NBT), 1-(p-jodofenyl)-5-(p-nitrofenyl)-3-fenylformazan (INT) and 3-(4, 5-dimethylthiazol-2-yl)-5-(3-carboxymethoxyphenyl)-2-(4-sulfophenyl)-2H-tetrazolium (MTS);
said reagent composition further comprises a mediator in the form of at least one of phenazine methosulphate (PMS) and 1-methoxy-5-methylphenazinium methylsulphate (mPMS);
said reagent composition further comprises lactate and NAD⁺, wherein said reagent composition is arranged to determine LDH in a buffer comprising N-methyl-D-glucamine;
the pH in the reagent composition is between 9-11.

81. The method according to claim 80, wherein the pH in the reagent composition is between 9.5-10.5.

82. The method according to claim 80, wherein the pH in the reagent composition is between 9.8-10.2.

83. The method according to claim 75, wherein said chemical means is a reagent composition in the form of dry chemical means or wet chemical means, and where said reagent composition is arranged to determine Hb and comprises a benzidine compound preferably selected from the group consisting of tetramethylbenzidine (TMB) and 3,3′-diaminobenzidine (DAB), wherein said reagent composition comprises a peroxide substrate preferably selected from the group consisting of hydrogen peroxide and tert-butylhydroperoxid (T-hydro) and wherein preferably the pH in the reagent composition is between 3-7.

84. The method according to claim 83, wherein the pH in the reagent composition is between 4.5-5.5.

85. The method according to claim 75, wherein the total amount of a prognostic marker, such as Hb and LDH respectively, in a sample of body fluid (9) is estimated by comparison to a standard reference scale of increasing color intensity, where absence of color or less intense color corresponds to low concentration of marker and more intense color corresponds to high concentration of marker.

86. The method according to claim 75, wherein a sample of body fluid (9) is collected from a newborn infant for assessing hypoxia and allowing for prediction of hypoxic ischemic encephalopathy after prenatal asphyxia.

87. The method according to claim 75, wherein the blood sample is collected prior to a medical procedure.

88. The method according to claim 87, where said medical procedure involves transplantation.

89. The method according to claim 87, where said medical procedure is surgery of the gastrointestinal tract.

90. Use of a disposable device for assessing hypoxia induced cellular damage in a mammal according to the method of claim 75, said device (2) comprising at least a sample inlet (4) and a collection chamber (32) arranged with a separation device (3), wherein the collection chamber (32) is connected to at least two, a first (5A) and a second (5B), visible detection compartments, each arranged with chemical means for direct detection, said first detection compartment (5A) being arranged to determine whether the amount of hemoglobin (Hb) in a sample of body fluid (9) from said mammal exceeds a predetermined level, and said second detection compartment (5B) being arranged to evaluate level of total amount of lactate dehydrogenase (LDH) in said sample.

91. The use according to claim 90, wherein the disposable device (2) comprises more than two visible detection compartments (5A-C) arranged on the card, preferably each one of said compartments arranged with chemical means in the form of a reagent composition.

92. The use according to claim 91, wherein each of said at least two visible detection compartments (5A-C) is arranged with reagent composition for direct visual detection of one member of the group consisting of prognostic markers: Hb, LDH, aspartate aminotransferase (AST), alanine aminotransferase (ALT), lactate, creatine kinase (CK), K, Mg and Ca.
